(19) United States
(12) Patent Application Publication
(10) Pub. No.: US 2019/0010099 A1
(43) Pub. Date: Jan. 10, 2019

(54) FRACTIONAL DISTILLATION SYSTEMS AND METHODS FOR LINEAR ALPHA OLEFIN PRODUCTION

(71) Applicant: SABIC Global Technologies B.V., Bergen op Zoom (NL)

(72) Inventors: Abdullah Saad Al-Dughaither, Riyadh (SA); Abdulmajeed Mohammed Al-Hamdan, Riyadh (SA); Shahid Azam, Riyadh (SA); Haresh Patel, Ahmedabad (IN); Ahmed Abdullah Hasanain, Jubail (SA)

(73) Assignee: SABIC Global Technologies B.V., Bergen op Zoom (NL)

(21) Appl. No.: 16/065,897

(22) PCT Filed: Dec. 23, 2016
(86) PCT No.: PCT/IB2016/057982

§ 371 Date: Jun. 25, 2018

Related U.S. Application Data
(60) Provisional application No. 62/272,354, filed on Dec. 29, 2015.

(51) Int. Cl. C07C 7/04; C07C 7/00; C07C 2/06; B01D 3/14

(52) U.S. Cl. CPC C07C 7/04; B01D 3/143; C07C 2/06; C07C 7/005

(57) ABSTRACT

A method for recovery of a product fraction from a mixture comprising an impurity and linear alpha olefins, the method comprising: introducing the mixture into a distillation column, wherein an overhead stream is separated from a bottom stream, wherein the overhead stream comprises a Cn product, a Cn+x product, and an impurity, wherein n is an integer from 4 to 30, wherein x is an integer from 0 to 26; directing the overhead stream into a fractionation column; and separating a Cn product fraction and a Cn+x product fraction in the fractionation column, wherein the Cn+x product fraction comprises at least a portion of the impurity, wherein, when an amount of the impurity is greater than 1 part per million in the Cn+x product fraction, at least a portion of the collected Cn+x product fraction is recycled to the distillation column.

### BACKGROUND

Linear olefins are one of the most useful classes of hydrocarbons used as raw materials in the petrochemical industry. Among these linear alpha-olefins, unbranched olefins whose double bond is located at a terminus of the chain, form an important subclass. Linear alpha olefins can be converted to linear primary alcohols by hydroformylation. Hydroformylation can also be used to prepare aldehydes as the major products which in turn can be oxidized to afford synthetic fatty acids, especially those with an odd carbon number, useful in the production of lubricants. Linear alpha olefins are also used in the most important class of detergents for domestic use, namely the linear alkylbenzene sulfonates, which are prepared by Fiedel-Crafts reaction of benzene with linear olefins followed by sulfonation.

Although linear olefins are the product of dehydrogenation of linear alkanes, the major portion of such products consists of the internal olefins. Preparation of alpha olefins is based largely on oligomerization of ethylene, which has a corollary that the alpha-olefins produced have an even number of carbon atoms. Oligomerization processes for ethylene are based mainly on organoaluminum compounds or transition metals as catalysts.

Oligomerization methods for preparing linear alpha-olefins are widely known in the art. These methods are typically carried out in the presence of a catalyst preferably comprising a zirconium component, such as zirconium tetraisobutyrate, and an aluminum component as activator, such as ethyl aluminum sesquichloride.

Typically, the effluent from the reactor used to produce the linear alpha olefins is directed to one or more distillation columns to separate the various fractions of linear alpha olefins. One problem associated with the recovery of various fractions of produced linear alpha olefins includes impurities, such as solvents and catalysts, contaminating the various fractions. For example, under certain conditions, such as during a plant startup or feed flow interruptions, the amount of impurities in the C₄ and C₆ streams may reach up to 10,000 parts per million. As a result, the isolated fractions are off-specification (i.e., do not meet market demands of purity) and cannot be used without further purification.

Therefore, there is a need for improved methods of recovering off-specification fractions of linear alpha olefins.

### SUMMARY

Disclosed herein are methods for recovering a product fraction from a mixture.

A method for recovery of a product fraction from a mixture comprising an impurity and linear alpha olefins, the method comprising: introducing the mixture into a distillation column, wherein an overhead stream is separated from a bottom stream, wherein the overhead stream comprises a Cn product, a Cn+x product, and an impurity, wherein n is an integer from 4 to 30, wherein x is an integer from 0 to 26; directing the overhead stream into a fractionation column; and separating a Cn product fraction and a Cn+x product fraction in the fractionation column, wherein the Cn+x product fraction comprises at least a portion of the impurity, wherein, when an amount of the impurity is greater than 1 part per million in the Cn+x product fraction, at least a portion of the collected Cn+x product fraction is recycled to the distillation column.

A method for recovery of a hexene-1 from a mixture comprising toluene and linear alpha olefins, the method comprising: introducing the mixture into a distillation column, wherein an overhead stream is separated from a bottom stream, wherein the overhead stream comprises butene-1, hexene-1, and an impurity; directing the overhead stream into a fractionation column; and separating the butene-1 and hexene-1 to yield a butene-1 fraction and a hexene-1 fraction, wherein the hexene-1 fraction comprises at least a portion of the impurity, wherein, when an amount of the impurity is greater than 1 part per million in the hexene-1 fraction, at least a portion of the collected hexene-1 fraction is recycled to the distillation column.

A method for producing linear alpha olefins, the method comprising: oligomerizing ethylene to produce linear alpha olefins, wherein the linear alpha olefins include hexene-1 and butene-1; introducing the linear alpha olefins into a distillation column, wherein an overhead stream is separated from a bottom stream, wherein the overhead stream comprises butene-1, hexene-1, and an impurity; and directing the overhead stream into a fractionation column, wherein the butene-1 and hexene-1, are separated to yield a butene-1 fraction and a hexene-1 fraction, wherein the hexene-1 fraction comprises at least a portion of the impurity, wherein, when an amount of the impurity is greater than 1 part per million in the hexene-1 fraction, at least a portion of the collected hexene-1 fraction is recycled to the distillation column.

The above described and other features are exemplified by the following figures and detailed description.

### BRIEF DESCRIPTION OF THE DRAWINGS

Refer now to the figures, which are exemplary embodiments, and wherein the like elements are numbered alike.

FIG. 1 is a schematic of an embodiment of the method and system disclosed herein.

### DETAILED DESCRIPTION

Disclosed herein are systems and methods for recovery of a product fraction from a mixture comprising an impurity and linear alpha olefins. As disclosed herein, a method for recovery of a product fraction from a mixture comprising an impurity and linear alpha olefins can include introducing the mixture into a distillation column. In the distillation column, an overhead stream can be separated from a bottom stream. The overhead stream can include a Cn product, a Cn+x product, and an impurity. N can be an integer from 4 to 30. X can be an integer from 0 to 26. In an embodiment, n can be equal to 4 and x can be equal to 2. In an embodiment, x can be equal to 4. The overhead stream can be directed into a fractionation column. The Cn product fraction and a Cn+x product fraction can be separated in the fractionation column. The Cn+x product fraction can include at least a portion of the impurity. When an amount of the impurity is greater than 1 part per million in the Cn+x product fraction, at least a portion of the collected Cn+x product fraction can be recycled to the distillation column.

The impurity in the overhead stream can include toluene, benzene, p-xylene, m-xylene, ethyl benzene, paraffins, naphthenes, cyclo-paraffins or a combination comprising at least one of the foregoing.

The bottom stream can include an impurity. For example, the impurity in the bottom stream can include toluene, benzene, p-xylene, m-xylene, ethyl benzene, paraffins, naphthenes, cyclo-paraffins or a combination comprising at least one of the foregoing.

When the amount of the impurity is greater than 1 part per million in the Cn+x product fraction, the Cn+1 product fraction can be directed to a collection tank before recycling to the distillation column.

As disclosed herein, a method for recovery of a hexene-1 from a mixture comprising toluene and linear alpha olefins can include introducing the mixture into a distillation column. In the distillation column, an overhead stream can be separated from a bottom stream. The overhead stream can include butene-1, hexene-1, and an impurity. The overhead stream can be directed into a fractionation column. The butene-1 and hexene-1 can be separated to yield a butene-1 fraction and a hexene-1 fraction. The hexene-1 fraction can include at least a portion of the impurity. When an amount of the impurity is greater than 1 part per million in the hexene-1 fraction, at least a portion of the collected hexene-1 fraction can be recycled to the distillation column.

The impurity can include at least one of toluene, benzene, p-xylene, m-xylene, and ethyl-benzene.

The overhead stream can include, octene-1, decene-1, or a combination comprising at least one of the foregoing.

The overhead stream can includes a C₁₂-C₂₀₊ linear alpha olefin.

In an embodiment, when the amount of the impurity is greater than 1 part per million in the hexene-1 fraction, the portion of the collected hexene-1 fraction can be directed to a collection tank before recycling to the distillation column. A capacity of the collection tank is greater than or equal to 350 cubic meters, for example, greater than or equal to 500 cubic meters, for example, greater than or equal to 750 cubic meters, for example, greater than or equal to 1,000 cubic meters, for example, greater than or equal to 1,500 cubic meters.

In an embodiment, when the amount of the impurity is greater than 1 part per million in the hexene-1 fraction, the portion of the collected hexene-1 fraction can be directed to the distillation column at a rate of 0.1 to 10 tons per hour, for example, 0.25 to 7 tons per hour, for example, 0.3 to 5 tons per hour, for example, 0.5 to 4 tons per hour, for example, 0.75 to 3 tons per hour. For example, when the amount of the impurity is greater than 1 part per million in the hexene-1 fraction, the portion of the collected hexene-1 fraction can be directed to the distillation column at a rate of 0.5 to 1.5 tons per hour.

In an embodiment, a method for producing linear alpha olefins can include oligomerizing ethylene to produce linear alpha olefins. The linear alpha olefins can include hexene-1 and butene-1. The linear alpha olefins can be introduced into a distillation column where an overhead stream can be separated from a bottom stream. The overhead stream can include butene-1, hexene-1, and an impurity. The overhead stream can be directed into a fractionation column. In the fractionation column, the butene-1 and hexene-1 can be separated to yield a butene-1 fraction and a hexene-1 fraction. The hexene-1 fraction can include at least a portion of the impurity. When amount of the impurity is greater than 1 part per million in the hexene-1 fraction, at least a portion of the collected hexene-1 fraction can be recycled to the distillation column.

The impurity can include at least one of toluene, benzene, p-xylene, m-xylene, ethyl-benzene, paraffins, naphthenes, and cyclo-paraffins.

The linear alpha olefins can include a C₁₂-C₂₀₊ linear alpha olefin.

The linear alpha olefins can include at least one of octene-land decene-1.

When the amount of the impurity is greater than 1 part per million in the hexene-1 fraction, the portion of the collected hexene-1 fraction can be directed to a hexene-1 collection tank before recycling to the distillation column.

When the amount of the impurity is greater than 1 part per million in the hexene-1 fraction, the portion of the collected hexene-1 fraction can be directed to the distillation column at a rate of 0.1 to 10 tons per hour, for example, 0.25 to 7 tons per hour, for example, 0.3 to 5 tons per hour, for example, 0.5 to 4 tons per hour, for example, 0.75 to 3 tons per hour. For example, when the amount of the impurity is greater than 1 part per million in the hexene-1 fraction, the portion of the collected hexene-1 fraction can be directed to the distillation column at a rate of 0.5 to 1.5 tons per hour.

1-Hexene is commonly manufactured by two general routes: (i) full-range processes via the oligomerization of ethylene and (ii) on-purpose technology. A minor route to 1-hexene, used commercially on smaller scales, is the dehydration of hexanol. Prior to the 1970s, 1-hexene was also manufactured by the thermal cracking of waxes. Linear internal hexenes were manufactured by chlorination/dehydrochlorination of linear paraffins.

“Ethylene oligomerization” combines ethylene molecules to produce linear alpha-olefins of various chain lengths with an even number of carbon atoms. This approach results in a distribution of alpha-olefins. Oligomerization of ethylene can produce 1-hexene.

Fischer-Tropsch synthesis to make fuels from synthesis gas derived from coal can recover 1-hexene from the aforementioned fuel streams, where the initial 1-hexene concentration cut can be 60% in a narrow distillation, with the remainder being vinylidenes, linear and branched internal olefins, linear and branched paraffins, alcohols, aldehydes, carboxylic acids, and aromatic compounds. The trimerization of ethylene by homogeneous catalysts has been demonstrated.

There are a wide range of applications for linear alpha olefins. The lower carbon numbers, 1-butene, 1-hexene and 1-octene can be used as comonomers in the production of polyethylene. High density polyethylene (HDPE) and linear low density polyethylene (LLDPE) can use approximately 2-4% and 8-10% of comonomers, respectively.

Another use of C₄-C₈ linear alpha olefins can be for production of linear aldehyde via oxo synthesis (hydroformylation) for later production of short-chain fatty acid, a carboxylic acid, by oxidation of an intermediate aldehyde, or linear alcohols for plasticizer application by hydrogenation of the aldehyde.

An application of 1-decene is in making polyalphaolefin synthetic lubricant base stock (PAO) and to make surfactants in a blend with higher linear alpha olefins.

C₁₀-C₁₄ linear alpha olefins can be used in making surfactants for aqueous detergent formulations. These carbon numbers can be reacted with benzene to make linear alkyl benzene (LAB), which can be further sulfonated to linear alkyl benzene sulfonate (LABS), a popular relatively low cost surfactant for household and industrial detergent applications.

Although some C₁₄ alpha olefin can be sold into aqueous detergent applications, C₁₄ has other applications such as being converted into chloroparaffins. A recent application of C₁₄ is as on-land drilling fluid base stock, replacing diesel or kerosene in that application. Although C₁₄ is more expensive than middle distillates, it has a significant advantage environmentally, being much more biodegradable and in handling the material, being much less irritating to skin and less toxic.

C₁₆-C₁₈ linear olefins find their primary application as the hydrophobes in oil-soluble surfactants and as lubricating fluids themselves. C₁₆-C₁₈ alpha or internal olefins are used as synthetic drilling fluid base for high value, primarily off-shore synthetic drilling fluids. The preferred materials for the synthetic drilling fluid application are linear internal olefins, which are primarily made by isomerizing linear alpha-olefins to an internal position. The higher internal olefins appear to form a more lubricious layer at the metal surface and are recognized as a better lubricant. Another application for C₁₆-C₁₈ olefins is in paper sizing. Linear alpha olefins are, once again, isomerized into linear internal olefins are then reacted with maleic anhydride to make an alkyl succinic anhydride (ASA), a popular paper sizing chemical.

C₂₀-C₃₀ linear alpha olefins production capacity can be 5-10% of the total production of a linear alpha olefin plant. These are used in a number of reactive and non-reactive applications, including as feedstocks to make heavy linear alkyl benzene (LAB) and low molecular weight polymers used to enhance properties of waxes.

The use of 1-hexene can be as a comonomer in production of polyethylene. High-density polyethylene (HDPE) and linear low-density polyethylene (LLDPE) use approximately 2-4% and 8-10% of comonomers, respectively.

Another use of 1-hexene is the production of the linear aldehyde heptanal via hydroformylation (oxo synthesis). Heptanal can be converted to the short-chain fatty acid heptanoic acid or the alcohol heptanol.

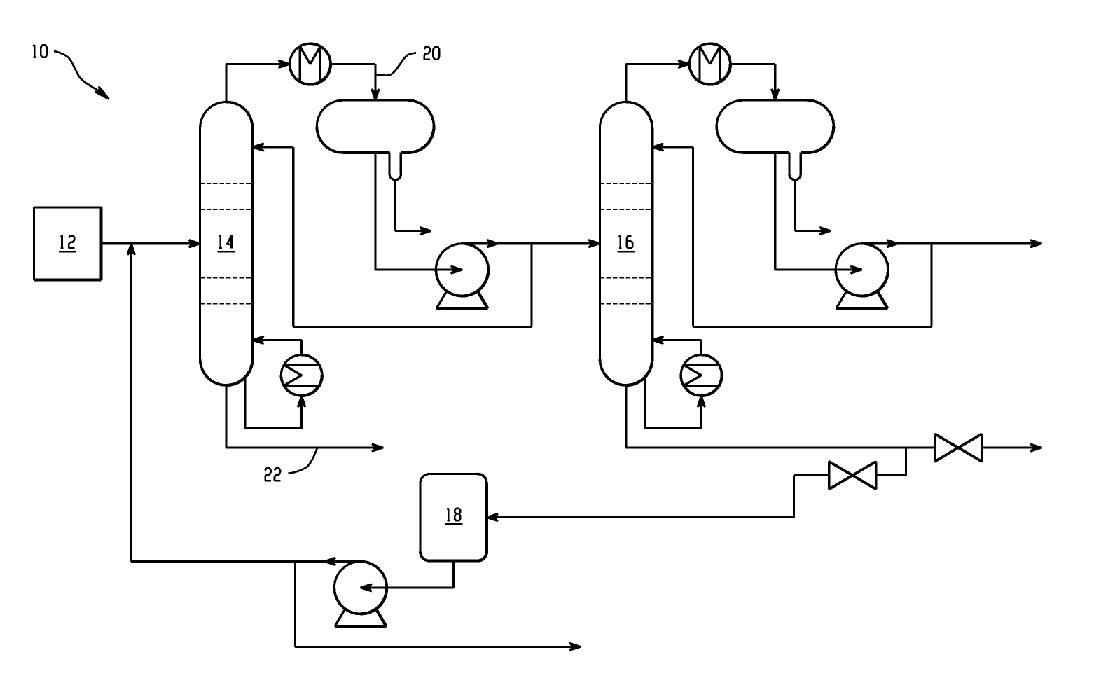

As shown in FIG. 1, the method 10 can include introducing a reaction mixture from a reactor 12 to a distillation column 14, wherein an overhead stream 20 is separated from a bottom stream 22. Solvent can be removed through the bottom stream 22. The overhead stream 20 can include a Cn product, a Cn+x product, and an impurity, wherein n can be an integer from 4 to 30 and wherein x can be an integer from 0 to 26. For example, the overhead stream can include a C₄ and a C₆ alpha olefin. In an example, the overhead stream 20 can include octene-1, decene-1, a C₁₂-C₂₀₊ linear alpha olefin, or a combination comprising at least one of the foregoing. The overhead stream 20 can be directed into a fractionation column 16 wherein a Cn product fraction and a Cn+x product fraction can be separated. Water can be removed before or concurrently when the overhead stream 20 is directed into the fractionation column 16.

However, during under unstable conditions, the overhead stream can include a high level of impurities (e.g., greater than 1 parts per million). For example, the impurities can include toluene, benzene, p-xylene, m-xylene, ethyl benzene, paraffins, naphthenes, cyclo-paraffins, or a combination comprising at least one of the foregoing. In an example, the Cn+x product fraction from the fractionation column 16 can include at least a portion of the impurity. In other words, the impurity can be isolated in the Cn+x product fraction, wherein the impurity is not present or, not significantly present in the Cn product fraction. For example, the C₆ alpha olefin product fraction can include the impurity of toluene. When an amount of the impurity is greater than 1 part per million in the Cn+x product fraction, at least a portion of the collected Cn+x product fraction can be recycled to the distillation column 14. In an example, the portion of the collected Cn+x product fraction can be stored in an off-spec collection tank 18 (e.g., a C6 off-spec collection tank) before being recycled to the distillation column 14. The collection tank 18 can be any desired size that will hold the impurities for an amount of time before being recycled to the distillation column 14. For example, the off spec collection tank 18 can be greater than or equal to 350 cubic meters, for example, greater than or equal to 500 cubic meters, for example, greater than or equal to 750 cubic meters, for example, greater than or equal to 1,000 cubic meters. The rate at which the collected Cn+x product fraction is directed to the distillation column 12 can be 0.1 to 10 tons per hour, for example, 0.25 to 7 tons per hour, for example, 0.3 to 5 tons per hour, for example, 0.5 to 4 tons per hour, for example, 0.75 to 3 tons per hour.

In an example, the overhead stream 20 can include butane-1, hexene-1, an impurity, or a combination comprising at least one of the foregoing. The butane-1 and hexene-1 can be separated in the fractionation column 16, wherein the hexene-1 product fraction can include the impurity. When the amount of impurity is greater than 1 part per million, then the hexene-1 fraction can be recycled to the off spec collection tank 18 or the hexane-1 fraction can be recycled directly to the distillation column 14.

A process simulation model has been developed based on the current design of the distillation columns in FIG. 1. For the proposed reprocessing rate (1 ton per hour), the corresponding increase in reflux ratio, reboiler, and condenser duties were calculated using the simulation model to assess the columns' capabilities to handle the reprocessing of off-spec C₆ fraction, as shown in Table 1. However, different reprocessing rates other than 1 ton/hour are contemplated by taking into account the time required to completely reprocess the inventory of the C₆ off-spec tank.

The C₆ flow rate under full load can be approximately 4 tons per hour. However, under unstable conditions, the C₆ flow rate is below the full load rate. The duration during which the C₆ stream is routed to the C₆ off-spec tank is dependent on how fast the distillation columns will resume normal operation.

The capacity of the C₆ off-spec tank can be within the range of 1,000 cubic meters. Therefore, with a reprocessing rate of 1 ton per hour, it can take approximately 28 days to fully reprocess the off-spec C₆ stored in the off-spec tank.

TABLE 1
Percentage Increase in Process Parameters

| | Distillation Column | Fractionation Column |
|---|---|---|
| Increase in Reflux Ratio | 6.13% | 14.84% |
| Increase in Reboiler Duty | 7.86% | 8.44% |
| Increase in Condenser Duty | 13.85% | 6.00% |

The systems and methods disclosed herein include at least the following embodiments:

Embodiment 1: A method for recovery of a product fraction from a mixture comprising an impurity and linear alpha olefins, the method comprising: introducing the mixture into a distillation column, wherein an overhead stream is separated from a bottom stream, wherein the overhead stream comprises a Cn product, a Cn+x product, and an impurity, wherein n is an integer from 4 to 30, wherein x is an integer from 0 to 26; directing the overhead stream into a fractionation column; and separating a Cn product fraction and a Cn+x product fraction in the fractionation column, wherein the Cn+x product fraction comprises at least a portion of the impurity, wherein, when an amount of the impurity is greater than 1 part per million in the Cn+x product fraction, at least a portion of the collected Cn+x product fraction is recycled to the distillation column.

Embodiment 2: The method of Embodiment 1, wherein the impurity comprises toluene, benzene, p-xylene, m-xylene, ethyl benzene, paraffins, naphthenes, cyclo-paraffins or a combination comprising at least one of the foregoing.

Embodiment 3: The method of any of Embodiments 1 or 2, wherein n equals 4 and x equals 2.

Embodiment 4: The method of any of Embodiments 1-3, wherein x equals 4.

Embodiment 5: The method of any of Embodiments 1-4, wherein, when the amount of the impurity is greater than 1 part per million in the Cn+x product fraction, the Cn+1 product fraction is directed to a collection tank before recycling to the distillation column.

Embodiment 6: A method for recovery of a hexene-1 from a mixture comprising toluene and linear alpha olefins, the method comprising: introducing the mixture into a distillation column, wherein an overhead stream is separated from a bottom stream, wherein the overhead stream comprises butene-1, hexene-1, and an impurity; directing the overhead stream into a fractionation column; and separating the butene-1 and hexene-1 to yield a butene-1 fraction and a hexene-1 fraction, wherein the hexene-1 fraction comprises at least a portion of the impurity, wherein, when an amount of the impurity is greater than 1 part per million in the hexene-1 fraction, at least a portion of the collected hexene-1 fraction is recycled to the distillation column.

Embodiment 7: The method of Embodiment 6, wherein the impurity includes at least one of toluene, benzene, p-xylene, m-xylene, and ethyl-benzene.

Embodiment 8: The method of any of Embodiments 6-7, wherein the overhead stream includes, octene-1, decene-1, or a combination comprising at least one of the foregoing.

Embodiment 9: The method of any of Embodiments 6-8, wherein the overhead stream includes a C₁₂-C₂₀₊ linear alpha olefin.

Embodiment 10: The method of any of Embodiments 6-9, wherein, when the amount of the impurity is greater than 1 part per million in the hexene-1 fraction, the portion of the collected hexene-1 fraction is directed to a collection tank before recycling to the distillation column.

Embodiment 11: The method of any of Embodiments 6-10, wherein a capacity of the collection tank is greater than or equal to 750 cubic meters.

Embodiment 12: The method of any of Embodiments 6-11, wherein, when the amount of the impurity is greater than 1 part per million in the hexene-1 fraction, the portion of the collected hexene-1 fraction is directed to the distillation column at a rate of 0.5 to 4 tons/hour.

Embodiment 13: The method of any of Embodiments 6-12, wherein, when the amount of the impurity is greater than 1 part per million in the hexene-1 fraction, the portion of the collected hexene-1 fraction is directed to the distillation column at a rate of 0.5 to 1.5 tons/hour.

Embodiment 14: A method for producing linear alpha olefins, the method comprising: oligomerizing ethylene to produce linear alpha olefins, wherein the linear alpha olefins include hexene-1 and butene-1;introducing the linear alpha olefins into a distillation column, wherein an overhead stream is separated from a bottom stream, wherein the overhead stream comprises butene-1, hexene-1, and an impurity; and directing the overhead stream into a fractionation column, wherein the butene-1 and hexene-1, are separated to yield a butene-1 fraction and a hexene-1 fraction, wherein the hexene-1 fraction comprises at least a portion of the impurity, wherein, when an amount of the impurity is greater than 1 part per million in the hexene-1 fraction, at least a portion of the collected hexene-1 fraction is recycled to the distillation column.

Embodiment 15: The method of Embodiment 14, wherein the impurity is at least one of toluene, benzene, p-xylene, m-xylene, ethyl-benzene, paraffins, naphthenes, and cyclo-paraffins.

Embodiment 16: The method of any of Embodiments 14-15, wherein the linear alpha olefins include a C₁₂-C₂₀₊ linear alpha olefin.

Embodiment 17: The method of any of Embodiments 14-16, wherein the linear alpha olefins include at least one of, octene-land decene-1.

Embodiment 18: The method of any of Embodiments 14-18, wherein, when the amount of the impurity is greater than 1 part per million in the hexene-1 fraction, the portion of the collected hexene-1 fraction is directed to a hexene-1 collection tank before recycling to the distillation column.

Embodiment 19: The method of any of Embodiments 14-19, wherein, when the amount of the impurity is greater than 1 part per million in the hexene-1 fraction, the portion of the collected hexene-1 fraction is directed to the distillation column at a rate of 0.5 to 4 tons/hour.

Embodiment 20: The method of any of Embodiments 14-20, wherein, when the amount of the impurity is greater than 1 part per million in the hexene-1 fraction, the portion of the collected hexene-1 fraction is directed to the distillation column at a rate of 0.5 to 1.5 tons/hour.

In general, the invention may alternately comprise, consist of, or consist essentially of, any appropriate components herein disclosed. The invention may additionally, or alternatively, be formulated so as to be devoid, or substantially free, of any components, materials, ingredients, adjuvants or species used in the prior art compositions or that are otherwise not necessary to the achievement of the function and/or objectives of the present invention.

All ranges disclosed herein are inclusive of the endpoints, and the endpoints are independently combinable with each other (e.g., ranges of “up to 25 wt. %, or, more specifically, 5 wt. % to 20 wt. %”, is inclusive of the endpoints and all intermediate values of the ranges of “5 wt. % to 25 wt. %,” etc.). “Combination” is inclusive of blends, mixtures, alloys, reaction products, and the like. Furthermore, the terms “first,” “second,” and the like, herein do not denote any order, quantity, or importance, but rather are used to denote one element from another. The terms “a” and “an” and “the” herein do not denote a limitation of quantity, and are to be construed to cover both the singular and the plural, unless otherwise indicated herein or clearly contradicted by context. The suffix “(s)” as used herein is intended to include both the singular and the plural of the term that it modifies, thereby including one or more of that term (e.g., the film(s) includes one or more films). Reference throughout the specification to “one embodiment”, “another embodiment”, “an embodiment”, and so forth, means that a particular element (e.g., feature, structure, and/or characteristic) described in connection with the embodiment is included in at least one embodiment described herein, and may or may not be present in other embodiments. In addition, it is to be understood that the described elements may be combined in any suitable manner in the various embodiments.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. A method for recovery of a product fraction from a mixture comprising an impurity and linear alpha olefins, the method comprising:
introducing the mixture into a distillation column, wherein an overhead stream is separated from a bottom stream, wherein the overhead stream comprises a Cn product, a Cn+x product, and an impurity, wherein n is an integer from 4 to 30, wherein x is an integer from 0 to 26;
directing the overhead stream into a fractionation column; and
separating a Cn product fraction and a Cn+x product fraction in the fractionation column, wherein the Cn+x product fraction comprises at least a portion of the impurity,
wherein, when an amount of the impurity is greater than 1 part per million in the Cn+x product fraction, at least a portion of the collected Cn+x product fraction is recycled to the distillation column.

2. The method of claim 1, wherein the impurity comprises toluene, benzene, p-xylene, m-xylene, ethyl benzene, paraffins, naphthenes, cyclo-paraffins or a combination comprising at least one of the foregoing.

3. The method of claim 1, wherein n equals 4 and x equals 2.

4. The method of claim 1, wherein x equals 4.

5. The method of claim 1, wherein, when the amount of the impurity is greater than 1 part per million in the Cn+x product fraction, the Cn+1 product fraction is directed to a collection tank before recycling to the distillation column.

6. A method for recovery of a hexene-1 from a mixture comprising toluene and linear alpha olefins, the method comprising:
introducing the mixture into a distillation column, wherein an overhead stream is separated from a bottom stream, wherein the overhead stream comprises butene-1, hexene-1, and an impurity;
directing the overhead stream into a fractionation column; and
separating the butene-1 and hexene-1 to yield a butene-1 fraction and a hexene-1 fraction, wherein the hexene-1 fraction comprises at least a portion of the impurity,
wherein, when an amount of the impurity is greater than 1 part per million in the hexene-1 fraction, at least a portion of the collected hexene-1 fraction is recycled to the distillation column.

7. The method of claim 6, wherein the impurity includes at least one of toluene, benzene, p-xylene, m-xylene, and ethyl-benzene.

8. The method of any claim 6, wherein the overhead stream includes, octene-1, decene-1, or a combination comprising at least one of the foregoing.

9. The method of claim 6, wherein the overhead stream includes a C₁₂-C₂₀₊ linear alpha olefin.

10. The method of claim 6, wherein, when the amount of the impurity is greater than 1 part per million in the hexene-1 fraction, the portion of the collected hexene-1 fraction is directed to a collection tank before recycling to the distillation column.

11. The method of claim 6, wherein a capacity of the collection tank is greater than or equal to 750 cubic meters.

12. The method of claim 6, wherein, when the amount of the impurity is greater than 1 part per million in the hexene-1 fraction, the portion of the collected hexene-1 fraction is directed to the distillation column at a rate of 0.5 to 4 tons/hour.

13. The method of claim 6, wherein, when the amount of the impurity is greater than 1 part per million in the hexene-1 fraction, the portion of the collected hexene-1 fraction is directed to the distillation column at a rate of 0.5 to 1.5 tons/hour.

14. A method for producing linear alpha olefins, the method comprising:
oligomerizing ethylene to produce linear alpha olefins, wherein the linear alpha olefins include hexene-1 and butene-1;
introducing the linear alpha olefins into a distillation column, wherein an overhead stream is separated from a bottom stream, wherein the overhead stream comprises butene-1, hexene-1, and an impurity; and
directing the overhead stream into a fractionation column, wherein the butene-1 and hexene-1, are separated to yield a butene-1 fraction and a hexene-1 fraction, wherein the hexene-1 fraction comprises at least a portion of the impurity,
wherein, when an amount of the impurity is greater than 1 part per million in the hexene-1 fraction, at least a portion of the collected hexene-1 fraction is recycled to the distillation column.

15. The method of claim 14, wherein the impurity is at least one of toluene, benzene, p-xylene, m-xylene, ethyl-benzene, paraffins, naphthenes, and cyclo-paraffins.

16. The method of claim 14, wherein the linear alpha olefins include a C₁₂-C₂₀₊ linear alpha olefin.

17. The method of claim 14, wherein the linear alpha olefins include at least one of, octene-1 and decene-1.

18. The method of claim 14, wherein, when the amount of the impurity is greater than 1 part per million in the hexene-1 fraction, the portion of the collected hexene-1 fraction is directed to a hexene-1 collection tank before recycling to the distillation column.

19. The method of claim 14, wherein, when the amount of the impurity is greater than 1 part per million in the hexene-1 fraction, the portion of the collected hexene-1 fraction is directed to the distillation column at a rate of 0.5 to 4 tons/hour.

20. The method of claim 14, wherein, when the amount of the impurity is greater than 1 part per million in the hexene-1 fraction, the portion of the collected hexene-1 fraction is directed to the distillation column at a rate of 0.5 to 1.5 tons/hour.
